(19) United States
(12) Patent Application Publication
(10) Pub. No.: US 2004/0138115 A1
(43) Pub. Date: Jul. 15, 2004

(54) Local pain-combating agent

(76) Inventors: Burghardt Wittig, Berlin (DE); Christoph Stein, Berlin (DE); Michael Schafer, Berlin (DE); Matthias Schroff, Berlin (DE); Claas Junghans, Berlin (DE); Sven Andres Konig Merediz, Madrid (ES)

Correspondence Address: NILS H. LJUNGMAN, ESQUIRE, NILS H. LJUNGMAN & ASSOCIATES, P.O. BOX 130, GREENSBURG, PA, 15601-0130, US

(21) Appl. No.: 10/646,620

(22) Filed: Aug. 22, 2003

Related U.S. Application Data
(63) Continuation-in-part of application No. PCT/DE02/00583, filed on Feb. 19, 2002.

(30) Foreign Application Priority Data
Feb. 24, 2001 (DE) 101 09 092.7

(51) Int. Cl. A61K 38/18; A61K 48/00

(52) U.S. Cl. 514/12; 514/44

(57) ABSTRACT

A remedy to suppress the sensation of pain, especially in chronic inflammatory diseases, by means of expressing endogenous neuroendocrine peptides at the site of inflammation. In particular, the invention relates to the expression of POMC or CRF from locally injected DNA expression constructs, preferably covalently peptide-modified expression constructs.

### CONTINUING APPLICATION DATA

This application is a Continuation-In-Part application of International Patent Application No. PCT/DE02/00583, filed on Feb. 19, 2002, which claims priority from Federal Republic of Germany Patent Application No. 101 09 092.7, filed on Feb. 24, 2001. International Patent Application No. PCT/DE02/00583 was pending as of the filing date of this application. The United States was an elected state in International Application No. PCT/DE02/00583.

### BACKGROUND OF THE INVENTION

1. Field of the Invention

The invention refers to a remedy for reducing the sensation of pain, especially in acute and chronic inflammatory disease, by means of expressing endogenous neuroendocrine peptides at the site of inflammation. The invention is useful in genetherapeutic treatment.

2. Background Information

Pain is a signal of the body indicating disease or injury. As such, it is ingenious and important, despite being subjectively uncomfortable. If pain becomes chronic, its role as a means of warning moves to the background. Chronic pain is a medically and socio-economically important problem. In Germany alone, there live 700,000 to 800,000 patients with pain, of which only 10% can be treated adequately. Among the most important causes of pain-related diseases are the group of rheumatic diseases, cancers, and pain of unclear etiology, such as back pain and headache without determined physiological cause. Rheumatic diseases are the cause of costs of about 15 billion Euro annually in Germany alone. Currently, about 40 million patients are suffering from arthritis in the USA, the caseload has been estimated to ascend to 60 million in the year 2020 (J Managed Care Pharm 1999: 414-419).

The treatment of the underlying condition that causes the chronic pain (an endeavor that in many cases fails due to a lack of understanding of the pathogenic mechanisms or lack of opportunities for therapeutic intervention) is complemented by the need to reduce pain effectively. The two classes of substances that are mainly employed in this endeavor are opiates and the so-called non-steroidal anti-inflammatory drugs (NSAIDs).

Both classes of substances are not without problems in their application, because of the real or subjectively feared addictive potential, possible depression of breathing function, sickness and sedation (opiates) or the potentially dramatic side effects mainly in the gastrointestinal area (ulcers, bleeding: NSAIDs). In addition to the problems caused by chronic pain, NSAID-treated patients incur a significantly increased probability of suffering from gastrointestinal problems as a consequence of their therapy.

Thus, the need for locally effective drugs (at the site of pain origin), without the mentioned central nervous or gastrointestinal side effects, is obvious.

Since about 1990 it is known that opioid receptors in the periphery contribute to pain reduction (antinociception) in inflamed tissue, and that the effective component of this antinociception is endogenous beta-endorphin (β-END) (Stein et al., Proc. Nat. Acad. Sci. USA 87, 5935-5939 (1990)). The phenomenon is limited to inflamed tissue in the model of the inflamed rat paw (Stein et al. J. Neuroscience 10, 1292-1298 (1990)). β-END is produced and released by lymphocytes in the inflamed tissue (Cabot et al. J. Clin. Invest. 100, 142-148 (1997)).

Local release of corticotropin-releasing factor (CRF) is a prerequisite of the pain reducing effect of β-END, and for Interleukin-1β, a role supporting the antinociceptive effect of β-END could be demonstrated (Schafer et al. Proc. Nat. Acad. Sci. USA 91, 4219-4223; ibid. 93, 6096-6100 (1996)). The antinociceptive effect of the leucocytes depends on the migration of β-END-releasing cells into the inflamed tissue (Machelska et al, Nature Medicine 4, 1425-1428 (1998)). The state of research is reviewed in Machelska and Stein, Current Opinion in Anaesthesiology 1999, 579-581.

Ishii et al. (Experimental Neurology 166, 90-98 (2000)) were able to demonstrate that implantation of β-END producing tumor cell lines in the subarachnoidal compartment leads to pain reduction in an animal model. Similar results were shown by Wu et al. (Neural Transplant Plast 4, 15-26 (1993)). It needs to be pointed out that these investigations were not conducted on animals with acutely inflamed tissue, and the implantation of genetically modified tumor cells into the central nervous systems indicates a relatively distant detachment from solutions to the problem that might be applicable to humans.

Feingold and Iadarola (Hum Gene Therapy 10(7):1251-7 (1999)) demonstrated the antinociceptive effect of β-END released by genetically modified adenoviruses that were injected into the subarachnoidal compartment and infected the meninges. This methodology was registered as a patent by Iadarola et al. (WO 0016800 A2). A viral means of gene transfer is described by Wilson et al. (Proc Natl Acad Sci U S A 96, 3211-6 (1999); Brain Res 792, 133-5 (1998)), who use herpesvirus as a vector for gene transfer into central nervous tissue.

β-END is a peptide comprised of 31 amino acids. It is encoded on exon 3 of the gene for pro-opiomelanocortin (POMC) and synthesized as part of a precursor peptide, from which the peptide hormones ACTH and gamma-lipotropin are also derived. The transcript length of POMC is different for central and peripheric tissues (Grauerholz et al., Peptides 19, 939-948 (1998)).

It has been shown already (Bauer M. et al., Abstract Sun27, 31th Conference 2000 INRC, Jul. 16th to Jul. 18th 2000, Seattle, Wash. USA), that cell cultures were transfected with β-endorphin (END) expressing constructs and subsequently, cell lysates of these transfected cells were injected into rat paws. Thereby, recombinant END, contaminated with cellular components such as proteins, membrane fragments, organelles and nucleic acids was injected into rat paws. This experiment served to fundamentally test whether the constructs were able to express END, if it was formed in cultured cells, in a fashion analogous to the biotechnological production of recombinant protein, but without the extensive purification, which is pharmaceutically required. This experiment was not suitable, however, to determine whether the application of isolated expression constructs by injection into inflamed tissues, the only application method tolerable from a technical point of view, had an antinociceptive effect.

### OBJECT OF THE INVENTION

Departing from this state of the art, the aim of the present invention is to make available an effective remedy for the reduction or suppression of pain sensation in mammals, especially in human beings.

### SUMMARY OF THE INVENTION

The distinguishing elements of claim 1 serve to reach this aim.

According to the invention, this aim is reached by a remedy that comprises expression constructs—with the provision that cells or cell lysates are excluded—for the local expression of neuroendocrine peptides or functional parts thereof. In particular the exclusion of expression constructs that were administered into cells for subsequent vaccination/injection or were contained in cell lysates, in favor of injecting them in isolated form as “naked DNA” or complexed to polymers, leads to surprising successes.

An essential aspect of the invention is the suppression of pain by the local expression of neuroendocrine peptides. Another essential aspect of the invention is to provide expression constructs suited to the local synthesis of neuroendocrine peptides, in particular β-Endorphin by suitable modifications of its precursor POMC as well as corticotropin-releasing factor.

According to the invention, a remedy for the reduction or suppression of pain sensation is provided, which comprises expression constructs for the expression of neuroendocrine peptides or functional parts thereof, with an emphasis on β-endorphin from modifications of its precursor POMC and/or corticotropin-releasing factor.

The remedy comprises, according to the invention, a gene construct that contains at least the immediate-early promoter of cytomegalic virus (CMV), the DNA sequence of the pro-opiomelanocortin gene (POMC), deleted of the coding regions for adrenocorticotropic hormone (ACTH) and beta-melanocyte stimulating hormone (β-MSH), which encodes at least once for β-endorphin, and a suitable polyadenylation sequence.

Besides the synthesis of β-END, which is advantageous for the suppression of pain, expression of the POMC sequence leads also to the synthesis of at least the tissue hormones adrenocorticotropic hormone (ACTH) and beta-melanocyte stimulating hormone (β-MSH) and the possibility cannot be excluded that this synthesis of “byproducts” proves to be disadvantageous in later clinical development expression cassettes were engineered, in which the sequences encoding ACTH and β-MSH were deleted and substituted by sequences encoding β-endorphin (examples 1.3, 1.4 and FIG. 4) . These expression cassettes also showed synthesis of β-END in a radioimmunoassay (FIGS. 2a and 2b).

Especially those expression cassettes, in which the sequences encoding ACTH and β-MSH were substituted by β-END encoding sequences (2×β-END and 3×β-END), constitute a preferred embodiment of the invention, since this expression result was not to be expected, and hence surprising. From the native POMC sequence, a polypeptide is translated that is cleaved by endogenous proteases into the physiologically active peptide hormones. Proteases chemically recognize the amino acid sequence context at the site of cleavage. Therefore, it is surprising that the substitution of the native parts of the POMC gene, which do not encode β-Endorphin, by more β-Endorphin encoding sequences, leads to an increased production of endorphin by cells expressing these constructs. It is especially surprising since it was not to be expected that the amino acid sequence, which was changed in comparison to the native sequence, would lead to faithful protease activity at the cleavage sites.

The inventive work is therefore contained in an exchange cloning by which the sequence tracts β-End—ACTH—β-MSH that occur in the POMC in sequential order several times, were exchanged or substituted by repetitive β-END sequences (ACTH>β-END and beta-MSH>β-END), so that as a result, the sequences for β-END—β-END—β-END repeat each other and are expressed. The invention therefore utilizes the native sequence.

An overview of the sequences mentioned in this application is given in the following table. The numbers of the sequences refer to their numbering in the sequence protocol:

| Sequence Number | characteristics |
|---|---|
| Seq. ID 1 | rPOMC 1× b-endorphin |
| Seq. ID 2 | rPOMC 3× b-endorphin |
| Seq. ID 3 | POMC Rat (rPOMC-WT) |
| Seq. ID 4 | rPOMC without b-endorphin |
| Seq. ID 5 | NLS from SV-40 |
| Seq. ID 6 | CRF Rat (rCRF) |
| Seq. ID 7 | rPOMC 2× b-endorphin |
| Seq. ID 8 | POMC human |
| Seq. ID 9 | b-Endorphin sequence rat |
| Seq. ID 10 | b-Endorphin sequence human |

According to the invention, another subject of the invention is a DNA sequence, comprising two of the β-endorphin encoding sequence tracts of the pro-opiomelanocortin gene (POMC), namely the sequence represented in Seq. ID 7 (rPOMC 2×β-END) and in the following:

atgccgagat tctgctacag tcgctcaggg gccctgctgc tggccctcct gcttcagacc 60

tccatagacg tgtggagctg gtgcctggag agcagccagt gccaggacct caccacggaa 120

agcaacctgc tggcttgcat ccgggcctgc agactcgacc tctcggcgga gacgcccgtg 180

tttccaggca acggagatga acagcccttg actgaaaatc cccggaagta cgtcatgggt 240

cacttccgct gggaccgctt cggcccgaga aacagcagca gtgctggcgg ctcagcgcag 300

aggcgtgcgg aggaagagac ggcgggggga gatggccgtc cggagccaag tccacgggag 360

ggcaagcgct acggcggctt catgacctcc gagaagagcc agacgcccct ggtgacgctc 420

ttcaagaacg ccatcatcaa gaacgtgcac aagaagggcc agaagcgcta cggcggcttc 480

atgacctccg agaagagcca gacgcccctg gtgacgctct tcaagaacgc catcatcaag 540

aacgtgcaca agaagggcca gtga 564

According to the invention, another subject of the application is a DNA sequence comprising three sequence tracts from the pro-opiomelanocortin gene (POMC) encoding β-endorphin, namely the sequence represented in Seq. ID 2 (rPOMC 3×β-END) and in the following:

atgccgagat tctgctacag tcgctcaggg gccctgctgc tggccctcct gcttcagacc 60

tccatagacg tgtggagctg gtgcctggag agcagccagt gccaggacct caccacggaa 120

agcaacctgc tggcttgcat ccgggcctgc agactcgacc tctcggcgga gacgcccgtg 180

tttccaggca acggagatga acagcccttg actgaaaatc cccggaagta cgtcatgggt 240

cacttccgct gggaccgctt cggcccgaga aacagcagca gtgctggcgg ctcagcgcag 300

aggcgtgcgg aggaagagac ggcgggggga gatggccgtc cggagccaag tccacgggag 360

ggcaagcgct acggcggctt catgacctcc gagaagagcc agacgcccct ggtgacgctc 420

ttcaagaacg ccatcatcaa gaacgtgcac aagaagggcc agaagcgcta cggcggcttc 480

atgacctccg agaagagcca gacgcccctg gtgacgctct tcaagaacgc catcatcaag 540

aacgtgcaca agaagggcca gaagcgctac ggcggcttca tgacctccga gaagagccag 600

acgcccctgg tgacgctctt caagaacgcc atcatcaaga acgtgcacaa gaagggccag 660

tga 663

Furthermore, according to the invention, another subject of the application is a human DNA sequence comprising the sequence tract from the pro-opiomelanocortin gene (POMC) encoding β-endorphin, namely the sequence represented in Seq. ID 8 (human POMC) and in the following:

atgccgagat cgtgctgcag ccgctcgggg gccctgttgc tggccttgct gcttcaggcc 60

tccatggaag tgcgtggctg gtgcctggag agcagccagt gtcaggacct caccacggaa 120

agcaacctgc tgaaggggat gggacaaaag aggcggtggc aagatcttag atgcccacga 180

gtgccaagaa agcaggtggg cagacctgcc tgtagggagg cctcgacgct tgacacgccc 240

gacactgtgc cctgtgtcct cggcgagtgc atccgggcct gcaagcccga cctctcggcc 300

gagactccca tgttcccggg aaatggcgac gagcagcctc tgaccgagaa cccccggaag 360

tacgtcatgg gccacttccg ctgggaccga ttcggccgcc gcaacagcag cagcagcggc 420

agcagcggcg cagggcagaa gcgcgaggac gtctcagcgg gcgaagactg cggcccgctg 480

cctgagggcg gccccgagcc ccgcagcgat ggtgccaagc cgggcccgcg cgagggcaag 540

cgctcctact ccatggagca cttccgctgg ggcaagccgg tgggcaagaa gcggcgccca 600

gtgaaggtgt accctaacgg cgccgaggac gagtcggccg aggccttccc cctggagttc 660

aagagggagc tgactggcca gcgactccgg gagggagatg gccccgacgg ccctgccgat 720

gacggcgcag gggcccaggc cgacctggag cacagcctgc tggtggcggc cgagaagaag 780

gacgagggcc cctacaggat ggagcacttc cgctggggca gcccgcccaa ggacaagcgc 840

tacggcggtt tcatgacctc cgagaagagc cagacgcccc tggtgacgct gttcaaaaac 900

gccatcatca agaacgccta caagaagggc gagtga 936

The sequence tract encoding β-endorphin in the pro-opiomelanocortin gene comprises the following sequence (refer to Seq. ID 9):

GGCT TCATGACC TCCGAGAAGA GCCAGACGCC CCTGGTGACG

CTCTTCAAGA ACGCCATCAT CAAGAACGTG CACAAGAAGG GCCAG

The human sequence (see Seq. ID 10):

GGTT TCATGACC TCCGAGAAGA GCCAGACGCC CCTGGTGACG

CTGTTCAAAA ACGCCATCAT CAAGAACGCC TACAAGAAGG GCGAG

differs from the rat sequence in only six bases (underlined).

A similar expression construct encoding the corticotropin releasing factor (CRF) is also subject of the invention.

The existence of β-endorphin can be proven as a radio-immunoassay (RIA) by inserting the complete sequence of the POMC gene of the rat (Seq. ID 3) in between the immediate early promoter of the cytomegalic virus (CMV-promoter) and a suitable polyadenylation sequence as part of an expression plasmid (FIG. 1), and subsequently transfecting this expression plasmid into cell culture cells derived from rats or humans (FIGS. 2a and 2b). A control expression cassette, into which a stop codon was inserted in front of the β-END DNA sequence, shows no expression of β-END in the RIA (FIG. 3, right bar).

Another aspect of the invention relates to vectors for the production of expression constructs. Here, the plasmids pMOK and pNOK were employed, which contain the DNA sequences or the sequence tract encoding β-endorphin (β-END) in different variations, namely multiply, but also the sequence encoding corticotropin releasing factor (CRF). rPOMC is a polyprotein, from which the actual peptide hormones (β-MSH, ACTH, β-endorphin) are cleaved by proteinases endogenous to the cell. By substituting the sequences of β-MSH and ACTH with β-endorphin, a higher expression of β-END is intended, and hence a greater reduction of pain. Multiple employment of the β-endorphin encoding sequence tract is meant to lead to increased transcription and subsequent expression of β-endorphin (see above).

Different expression constructs were engineered from the plasmid pMOK-POMC. The details for the production are disclosed in EP 0 941 318 B1 and DE 198 26 758. Specifically, plasmid DNA, linear covalently closed unmodified (so-called “MIDGE”-) expression constructs, as well as MIDGE constructs modified with a peptide comprising the nuclear localization sequence (NLS) of the large T antigen of SV40 (MIDGE-NLS) were produced (FIG. 4). This modification shows the surprising advantage that such expression constructs significantly improve transfection efficacy. Coupling of the NLS sequence to the minimalistic expression constructs leads to a remarkable increase of gene transfer, and hence of expression of β-END. This increment of expression is attained by ligating, to the gene that is to be expressed, oligodesoxyribonucleotides to which a nuclear localization signal (NLS) had been covalently coupled before. As is known from in-vitro experiments, this sequence derived from the virus SV40 can facilitate the transport of DNA from the cytosol of the cell into the nucleus, and hence increase the transcription rate (see WO 00/37659).

Peptides conjugated to linear, covalently closed expression constructs are also fundamentally preferred. Preferred are cationic peptides of a length of between eight and twenty amino acids.

The injection of plasmids that contain the expression cassette for POMC into inflamed rat paws already leads to a significant decrease in the sensation of pain in the treated animals (see FIG. 3). This effect can be enhanced significantly by complexing the DNA with polyethyleneimine (PEI) (see FIG. 5). Complexation of DNA with positively charged macromolecules facilitates the uptake of DNA, furthermore, a kind of nuclear transport mechanism is being discussed (Chemin I.et al.,J. Viral Hepat, Nov; 5 (6): 369-75, 1998).

Injection of POMC encoding MIDGE also leads to a significant reduction of pain sensation. The injection of peptide-modified constructs (MIDGE-NLS), however, by far has the greatest effect (see FIG. 6).

Consequently, the use of the remedy according to the invention, comprising the inventive expression constructs, as a medical drug, especially as a vaccine, is claimed.

Particularly, the invention has the following advantages. The remedy according to the invention enables the treatment of chronic pain diseases, in which the use of opiates and non-steroidal inflammatory drugs (NSAIDs) is dispensed with. The known side effects that are associated with such drugs, such as sickness, depression of breathing function, addiction and dependency problems (with opiates), ulcers etc. can be avoided. Furthermore, the remedy according to the invention shows the surprising advantage of longer duration of the effect, namely in the order of days, compared to the injection of recombinant β-END (duration of the effect in the range of minutes).

The current invention therefore provides for a remedy for the suppression of pain, where—in contrast to Bauer M. et al. (Abstract Sun27, 31th Conference 2000 INRC, Jul. 16th to Jul. 18th 2000, Seattle, Wash. USA)—no transfection of DNA expression constructs into cell culture cells and subsequent use of the crude protein suspension takes place, but rather an injection of expressable constructs directly into the inflamed tissue for pain suppression. Precisely this effect could not be demonstrated by Bauer et al. No transfected cells or cell lysates are thus being used as component of the resulting vaccine. The avoidance of cell lysates leads to surprising successes, as is shown in more detail in the following. Surprisingly, the effect was greater with MIDGE compared to plasmid, and still much greater with MIDGE-NLS. This effect in particular is not observed with the use of cell lysates.

The above-discussed embodiments of the present invention will be described further hereinbelow. When the word “invention” is used in this specification, the word “invention” includes “inventions”, that is the plural of “invention”. By stating “invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Further advantageous measures are described in the other dependent claims; the invention is described in form of examples and the following figures:

FIG. 1 shows the functional composition of the employed expression plasmids, pMOK and pNOK, and the cloned sequences encoding rPOMC. The artificial sequences rPOMC-1βEND, rPOMC-2βEnd and rPOMC-3βEnd were recombined into the plasmid pNOK, which comprises a simple CMV promoter and no intron.

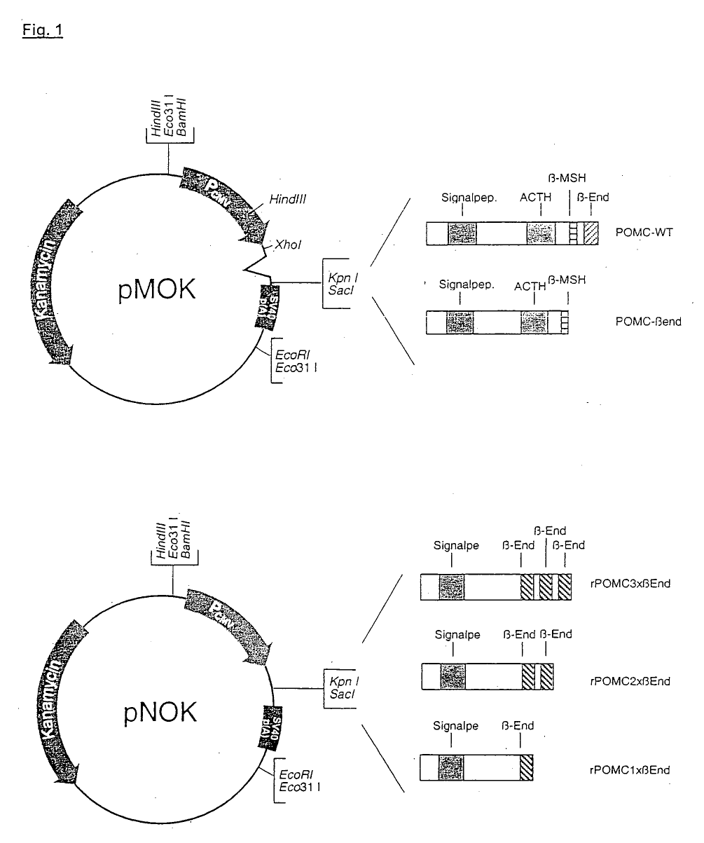

FIG. 2 shows the proof of expression of β-END in cell lysate of rat cell cultures by means of radioimmunoassay (RIA). All expression constructs showed a significantly higher expression of β-END. This result of increased expression from the artificial constructs was not to be expected and is in contrast to Bauer M. et al. (Abstract Sun27, 31th Conference 2000 INRC, Jul. 16th to Jul. 18th 2000, Seattle, Wash. USA), where only the naturally occurring POMC was employed. The results demonstrated, however, that expression after insertion into the pMOK vector (FIG. 2b) is more effective compared to insertion into the pNOK vector (FIG. 2a).

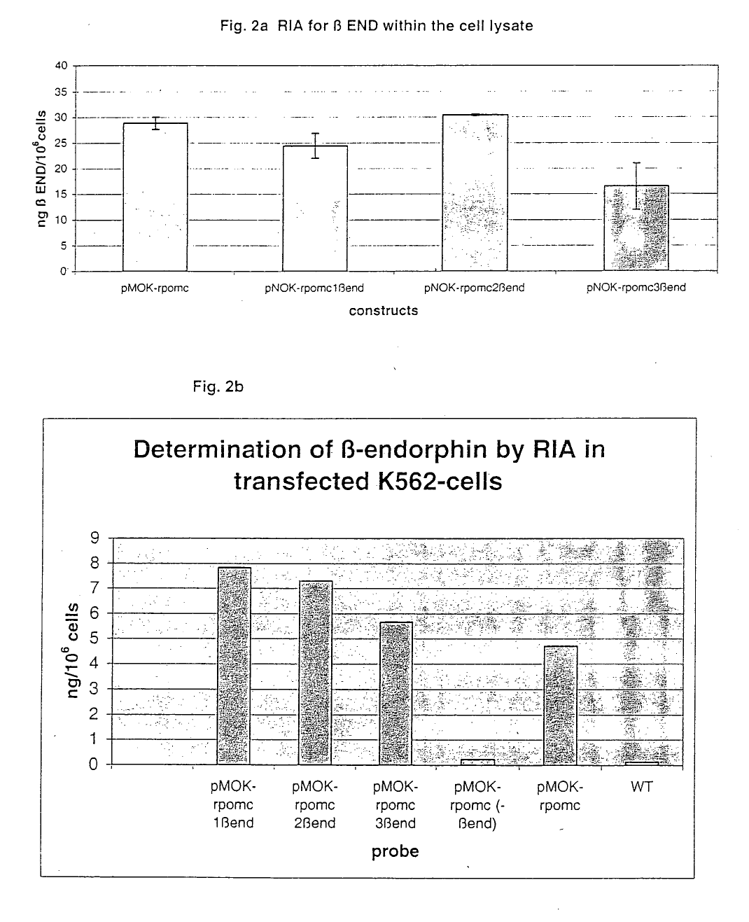

FIG. 3 shows the linear correlation of the amount of plasmid pMOK-rPOMC applied and the pain sensation threshold of the experimental animals. As plasmid insert, the complete rPOMC sequence was employed (example 1.1), and the plasmid was injected into the inflamed site in varying concentrations. To prove the pain reducing effect of β-END, an expression cassette lacking the expression cassette for β-END (Example 1.2) was made. As was expected, no pain reducing effect can be observed in the experiment, see the far right bar in FIG. 3.

FIG. 4 shows the different expression cassettes that were employed in the production of MIDGE constructs. The constructs so produced contain only the promoter and terminator sequence necessary for their expression. The coupling of peptides for nuclear localization (NLS sequence) was performed where desired.

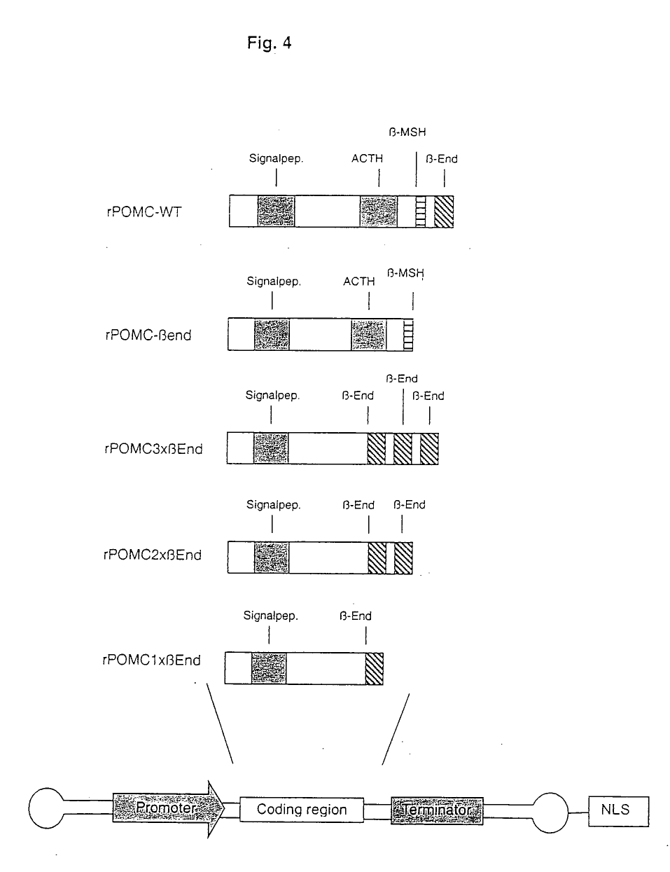

FIG. 5 shows the significant amplification of the expression of β-END by complexation of the DNA with PEI in the case of plasmid.

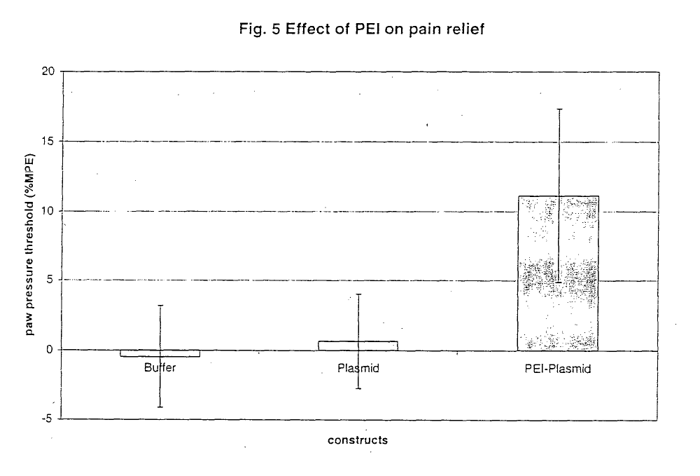

FIG. 6 shows the comparison of the antinociceptive effect of the expression constructs plasmid, MIDGE and MIDGE linked to NLS (MIDGE-NLS). Here, MIDGE-NLS is shown to be the most effective expression system by far.

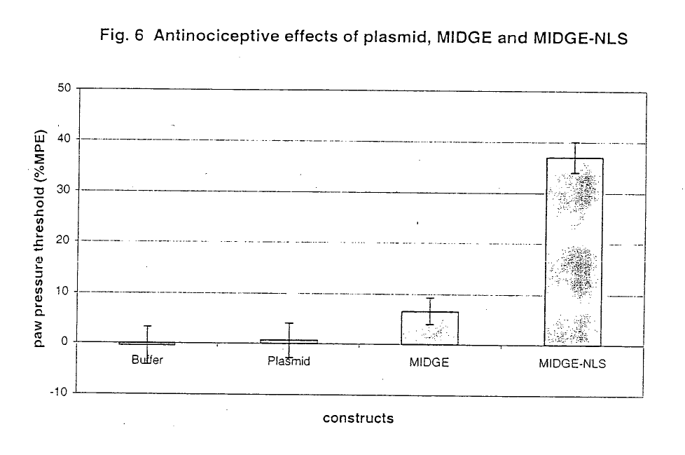

FIG. 7 shows the antinociceptive effect of peptide-modified MIDGE vectors in relation to time. A significant effect of pain reduction is even discernible after 96 hours, with applied concentrations of MIDGE-rPOMC-NLS at 50 and 125 mg.

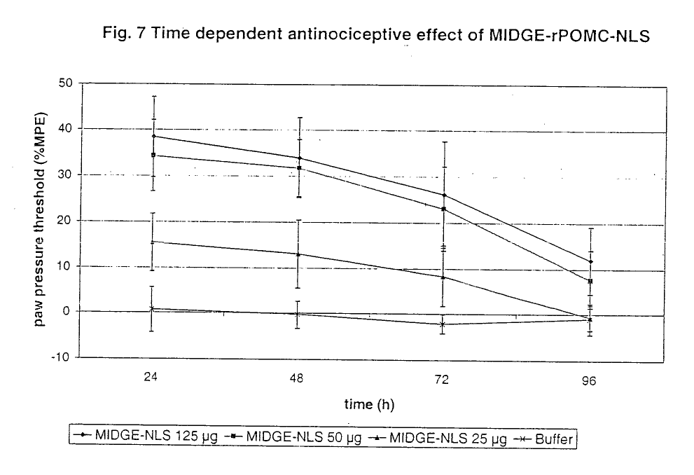

FIG. 8 shows a graphical representation of MIDGE synthesis.

FIG. 9 shows MIDGE allows for the attachment of tissue-specific ligands for the active uptake of the entire vector molecule by receptor internalization into the cell cytoplasm.

### DESCRIPTION OF THE PREFERRED EMBODIMENT

### EXAMPLE 1.1

### Cloning of rPOMC of the rat

RNA was isolated from rat brain cells and transcribed to DNA by means of reverse transcriptase and universal primers. With primers specific for the cDNA of POMC (left primer 5′-AATTATGGTACCATGCCGAGATTCTGCTACAG; right primer 5′-TTCTCAGAGCTCTCACTGGCCCTTCTTGTGCACGTTCTTGATG) a PCR was performed. The PCR product thus formed in the expected length was purified, digested with the restriction enzymes KpnI and SaeI and inserted into the vector pMOK. Clones were analyzed by restriction digest and clones of expected fragment length were confirmed by sequence analysis. The wild type sequence is given in Seq. ID 3 (rPOMC-WT).

### EXAMPLE 1.2

### Cloning of rPOMC-β-END

The plasmid pMOK-rPOMC served the template for the cloning of POMC-β-END. A fragment was amplified by PCR, which was truncated and lacked the sequence encoding β-END. The stop codon for the novel sequence was introduced in the course of the PCR reaction (the stop codon is marked in fat type in the right primer). The following primers were employed:

left primer:

5′-AATTATGGTACCATGCCGAGATTCTGCTACAG

right primer:

5′-ATTATGAGCTCTCAGCGCTTGTCCTTGGGCGGGTTG.

After purification of the PCR product and restriction digestion with KpnI and SacI, the fragment was inserted into the vector pMOK. Clones were analyzed by restriction digest and clones of expected fragment length were confirmed by sequence analysis. The sequence is given in Seq. ID 4 (rPOMC-β-END).

### EXAMPLE 1.3

### Cloning of rPOMC 1×β-END

The plasmid pMOK-rPOMC served the template for the cloning of POMC 1×β-END. The artificial gene construct is composed of the nucleotides of the POMC sequence until the last codon in front of the start of the ACTH sequence and the β-END sequence. For the joining of the gene sequence, two PCR reactions were necessary. The following primers were employed:

Fragment 1:

left primer:

5′-AATTATGGTACCATGCCGAGATTCTGCTACAG

right primer:

5′-ATTATTGAGCTCTAGAAGACATGCGCTTGCCCTCCCGTGGA

Fragment 2:

left primer:

5 ′-AATTATGGTCTCTGCGCTACGGCGGCTTCATGACCTC

right primer:

5′-AATTATGAGCTCTGAAGACATGCGCTTCTGGCCCTTCTTGTGCACGTTC

After amplification of the first fragment by PCR and subsequent purification, the fragment was digested by KpnI and SadI and inserted into the vector pMOK. Correct clones were identified by restriction digestion and confirmed by sequence analysis. The resulting plasmid was digested with BbsI and SacI. Fragment 2 was digested with BbsI and SacI. The overhanging ends generated by BbsI and SacI are complementary to one another. Fragment 2 was cloned into the vector that contained fragment 1, correct clones were identified by restriction digestion and confirmed by sequence analysis. The sequence is given in Seq. ID 1 (rPOMC 1×β-END).

### EXAMPLE 1.4

### Cloning of rPOMC 3×β-END

rPOMC also served as the template for the cloning of POMC 3×β-END. In comparison to fragment 2 of example 1.3, the sequence did not contain a stop codon at the end of the β-END sequence. The intermediate product and the fragment of example 1.3 could be employed here. The following primers were used for the amplification of fragment 3:

Fragment 3:

left primer:

5′-AATTATGGTCTCTGCGCTACGGCGGCTTCATGACCTC

right primer:

5 ′-TTCTCAGAGCTCTCACTGGCCCTTCTTGTGCACGTTCTTGATG.

After purification, fragment 3 was digested with Eco31I and SacI, and inserted into the intermediate product that had been digested with BbsI and SacI. The resulting intermediate product 2 was also digested with BbsI and SacI, and fragment 3 was inserted once more into the intermediate product 2. Intermediate product 3 was digested once more with BbsI and SacI and the fragment 2 from example 1.3 (this fragment contains the stop codon) was inserted as the last fragment into the intermediate product 3. The resulting plasmid contained 3 β-END sequences in sequence. The sequence is given in Seq. ID 2 (rPOMC 3×β-END).

### EXAMPLE 1.5

### Cloning of rPOMC-CRF

RNA was isolated from rat brain cells and transcribed to DNA by means of reverse transcriptase and universal primers. A PCR was performed with primers specific for the cDNA of the corticotropin releasing factor ((CRF); left primer: 5′-TTAATAGGTACCATGCGGCTGCGGCTGCTG; right primer: 5′-ATTATGAGCTCTCATTTCCCGATAATCTCCATC). The PCR product thus formed in the expected length was purified, digested with the restriction enzymes KpnI and SacI and inserted into the vector pMOK. Clones were analyzed by restriction digest and clones of correct fragment length were confirmed by sequence analysis. The sequence is given in Seq. ID 6 (rPOMC-CRF).

### EXAMPLE 1.6

### Production of MIDGE-rPOMC with and without NLS

MIDGE vectors are linear, covalently closed expression cassettes, that only consist of a CMV promoter, an intron, the corresponding gene sequence and a polyadenylation sequence (see EP 0 941 318 B1). The constructs were obtained as follows: The plasmid pMOK-rPOMC described under example 1.1 was digested to completion by Eco31I. Ligation with 5′ phosphorylated hairpin-shaped oligodesoxynucleotides (ODN) 5′-AGGGGTCCAG-TTTTCTGGAC-3′ with T4 DNA Ligase in the presence of Eco31I was stopped by heating to 70° C. The resulting mixture was concentrated and treated with Eco31I and T7 DNA Polymerase in the absence of desoxyribonucleotide triphosphates. Separation was performed by anion exchange chromatography.

MIDGE vectors with NLS coupling were constructed as follows: the NLS peptide PKKKRKVEDPYC was linked to the ODN in two steps. Firstly, the modified oligonucleotide 5′-PH-d(GGGAGTCCAGT xT TTCTGGAC, where xT signifies the amino modified thymine base with a C₂—amino linker) (0,1 mM) was activated by sulfo-KMUS (5 mM) in PBS at room temperature. After 120 min, the reaction was stopped by addition of 50 mM tris-(hydroxymethyl)-aminomethane, and the activated ODN was obtained after ethanol precipitation, (300 mM NaOAc pH 5.2, 5.5 mM MgCl₂, 70% Ethanol), centrifugation and one rinse with 70% ethanol. The ODN thus obtained was dissolved in PBS and submitted to reaction with the peptide (0,2 mM) for one hour at room temperature. The reaction was checked by gel electrophoresis (3%) and ethidium staining. The NLS-linked ODN thus formed was purified by HPLC and employed in the synthesis of the MIDGE-POMC-NLS constructs as described above.

### EXAMPLE 2.1

### Pain reduction after injection of expression constructs for POMC

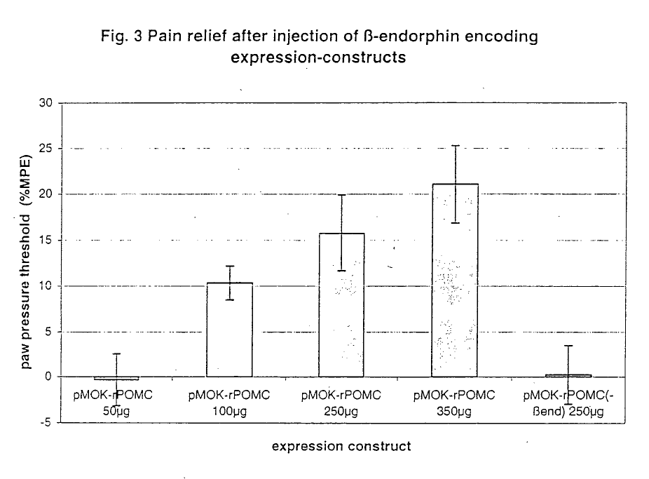

50, 100, 250 and 350 μg pMOK-rPOMC in a volume of 200 μl 150 mM sodium phosphate, pH 7,2 were injected into a inflamed rat hind paw. 24 h later, pain reduction in the inflamed rat paw was assayed. The “paw pressure threshold” method employed in this experiment is described in Schaefer et al., Proc. Nat. Acad. Sci USA 914219-4223 (1994) on page 4220 in the methods section. FIG. 3 shows the result of the experiment. Six animals per group were employed.

MIDGE Transfection Vector

The following section is a discussion of the MIDGE Transfection Vector, based on information published on the Internet by MOLOGEN Forschungs-, Entwicklungs- und Vertriebs GmbH.

Introduction—Gene therapy and genetic vaccination:

Gene therapy and genetic vaccination are new concepts for healing or preventing human diseases.

Most diseases have to do with genes at some level. Sometimes the genes are that of a foreign organism trying to invade, as in a virus or a bacteria-born disease such as tuberculosis, or even malaria. In other conditions, it might be the body's own genes that cease to work together as they were designed to, leading to cancer or autoimmune disease. Genetic diseases in the classic sense, meaning a defect in a gene leading to problems early or later in life, are relatively rare but sometimes very disabling to those afflicted by them.

In gene therapy, gene transfer is used to improve the condition of the patient. In some disorders, this shall be achieved by replacing or supplementing defective or missing genes.

In a wider sense, gene therapy also involves the concept of genetic immune modulation, for instance to suppress the rejection reaction after a transplantation, to suppress unfavorable immune stimulation in autoimmune diseases such as lupus, or to elicit a powerful immune response against a tumor.

In genetic vaccination, a gene encoding an antigenic protein cloned in expression vectors is transferred into the patient. The patient subsequently develops an immunological reaction against the bug, never having been exposed to more than the genetic blueprint of it.

The administration of genetic information to a patient can offer significant advantages over a more traditional pharmaceutical approach. Among these advantages are an increased efficacy, safety and cost-effectiveness of the treatment.

Vectors:

One of the key problems in gene therapy and genetic vaccination is finding a suitable transfer agent to carry the gene to be transferred to appropriate cells, and then obtaining sufficient levels of expression. Gene carriers are termed vectors. The lack of efficient vectors is the major problem concerning the medical use of genetic information.

There are a number of requirements of a vector that can be used in an in-vivo application:

tissue specificity—the vectors should have the ability to target specific cell types. This is important if the vector is given systemically to the patient via syringe or similar devices.

size of inserted DNA—an unlimited amount of inserted DNA should be accomodated by the vector.

non-immunogenic—if the vector itself elicits an immune response against itself or the transfected cell into which it has delivered its payload, transgene expression may be not sufficient. Moreover, an immunogenic vector may lead to adverse reactions in the patient.

stability—the vector must be stable against degradation by serum nucleases, exonucleases.

small size—to facilitate gene transfer into the cell and into the nucleus it is useful to have a vector that is as small as possible. At the same time the vector must be able to carry large genes if necessary.

nuclear transfer—attachment of nuclear localization sequences (NLS) must be possible to achieve a greater transfection efficacy into the nucleus.

easy to manufacture, stable as drug—for the commercial use, manufacture, storage, transport and distribution must be practical and relatively inexpensive

In the following, the most widely used vectors for clinical gene transfer are introduced and their main features are briefly discussed.

Viral Vectors:

A virus contains genetic information but cannot reproduce itself. To replicate, it must invade another cell and use parts of that cell's reproductive machinery. The elaborated systems for gene transfer and gene expression used by viruses constitute powerful tools for the construction of transfection vectors. Thus animal viruses have been used for gene transfer to mammalian cells.

In general, a virus is per se immunogenic, so are viral vectors.

Retroviral vectors—Retroviruses are RNA viruses which replicate through a DNA intermediate. The viral DNA integrates into the host genome. The viral genes required for replication are removed from the retrovirus and replaced by the therapeutic gene. Only regulatory elements remain of the original virus genome. Retroviral vectors are most frequently based upon the Moloney murine leukaemia virus (Mo-MuLV). The AIDS virus (HIV) is also a retrovirus.

Retroviral vectors enter the target cells, transcribe their RNA into DNA and integrate stably into the host genome, sometimes with very high efficiency.

A disadvantage of the murine retroviral vectors is their size limitations for the transgene. Retroviral integration and expression of viral genes requires that the target cells should be dividing. This limits gene therapy to mitotic cells. Another issue is the potential for producing recombinant viruses that can replicate, thus posing a severe threat of the patients safety. Furthermore, MuLV carry proto-oncogenes, which when mutated can induce oncogenesis (reviewed in Gray).

Lastly, using retrovirus is quite expensive, largely because of the need to test for contaminating virus, bacteria, and fungus. Particularly, the virus' envelope makes purification difficult and costly.

Adenovirus—Adenoviruses are the second most popular choice of viral gene delivery vectors. There are over forty serotype strains of adenovirus; however serotype 2 or 5 are predominantly used as vectors.

Adenoviral vectors are very efficient at transducing target cells and delivering its genetic cargo to the nucleus (Shenk, 1996). Viral replication occurs without integration into the host genome, leading to transient expression of the transferred gene (Verma&Somia). Adenovirus can be used for a wide range of cells, dividing and non-dividing.

The main disadvantage of the adenovirus is its immunogenicity, provoking the host immune system to attack the virus and render the therapy ineffective. Furthermore, adenovirus has been shown to cause damage in the brain and other organs. Adenovirus was used in the gene therapy study which led to the death of a teenager in September 1999.

Adeno-associated viral vector—Adeno-associated virus is a member of the parvovirus family that requires adeno or herpes virus for replication.

The adeno-associated virus is a single-stranded DNA virus, which combines the advantages of retroviral and adenoviral vectors. It has little immunogenicity and can infect a wide range of cell types, including both dividing and non-dividing cells.

In the absence of a helper virus, AAV integrates into the host genome on the short arme of chromosome 19 (Rivadeneira, 1998).

The main problems is that AAV vectors can only transport rather small genes, 5 kb is the upper limit (Smith, 1995). And, as with all viral vectors, there exists the danger of insertional mutagenesis and the generation of replication competent virus.

Non-viral Vectors:

The issue of immunogenicity of viral vectors, the possibility of recombination as well as their relatively small capacity for therapeutic DNA have led to the development of non-viral vectors. These vectors are using naked DNA encoding a protein sequence and the necessary regulatory elements to express it, which is carried by different techniques to the cells, resulting in endogenous synthesis of the encoded protein.

The ease of genetic manipulation of naked DNA vectors invites their use in gene therapy and genetic immunization. Using naked DNA as vector is also usually much cheaper than using viral vectors.

With non-viral vectors, the DNA is not stably integrated within the chromosomal DNA, but persists as extrachromosomal nuclear episomes.

Plasmids—Plasmids are useful for delivering large genes to many types of cells for short-term expression of the delivered gene.

A plasmid is a small piece of DNA, which can exist in a cell separate from the cell's main DNA. Plasmids are used extensively in biotechnology as a basis for transmitting genetic information from one organism into another, which serves as a host cell. A DNA segment of interest can be recombined into a plasmid, and then the plasmid can be transported into the host cells, which will divide, replicating the foreign DNA along with the host cells' DNA. Genes within the plasmid have various functions; one of the most useful is the ability to confer antibiotic resistance to the bacterium. Plasmids are used to replicate genes and also as vectors in gene delivery. Plasmids used as vectors contain the following essential elements(Donnelly, 1999):

a strong eukaryotic promoter such as the immediate early promoter from cytomegalovirus

a gene of interest

a transcription terminator such as that from bovine growth hormone

an antibiotic resistance gene or other selectable marker to facilitate selection of transformed organisms carrying the plasmid

an origin of replication, to allow for production of the plasmid in the desired host, that is not active in mammalian cells

Most commonly, plasmids of this type are produced in E. coli, using as a backbone an E. coli plasmid such as pBR322 or pUC18.

Plasmids have the advantage, compared to viral vectors, that they are easy to produce in large amounts and high purity at relatively low costs. Although they may be several magnitudes less efficient in their capacity to deliver transgenes into the nucleus than wild type virus in vivo, they do not raise the specter of infectivity or systemic reactions against the vector.

Plasmids have, however, a number of disadvantages. Conventional plasmid vectors carry elements that are not contributing to the targeted, focused delivery of the expressed genetic information. This is the dominating fraction of non-medical sequence content used only for the propagation of the vector during its production in bacteria. These sequences include, as noted above, antibiotic resistance and other selectable markers, the occurrence of which is coming under increased scrutiny by public health scientists even before their mass appearance in medical formulations.

Apart from the obvious argument of antibiotic resistance genes, the plasmid vector sequence content raises other concerns. Introduction into the body of DNA sequences as a chemical entity, rather than as coding information, can have dramatic physiological consequences. The well-published emerging field of immuno-stimulatory sequences (ISS), mainly unmethylated CG in specific sequence contents, are the most prominent members of this growing family (Krieg 1995). ISS were initially discovered when varying plasmid vectors in an immunization protocol led to drastically different results. The molecular mechanism of ISS action has since become a hot subject of research. One unifying feature of all results seems to be the induction of a powerful immune reaction of the IFN gamma/CTL type and of NK cells. Transcription of transgenes under control of viral promoters can be down-regulated in the cells subject to the ISS response, probably as a result of the molecular responses to ISS, i.e. the induction of IFN gamma. Consequentially, the presence of ISS is counterproductive to the expression of proteins (Tan 1999). Whatever fascinating opportunities ISS may offer, it can be assumed that the exclusion of ISS from expression vectors is beneficial to the objective of delivering a maximum of expression. Another much more worrying topic is the possibility of ISS sequences breaking tolerance to autoantigenes. The strong induction of CTL responses as a result of ISS administration will probably lead to a much-increased awareness of the immunological effects of vectors.

The MIDGE transfection vector technology:

Scientists at Mologen have developed a transfection vector which combines the advantages of viral-vectors (cell-specifity and high expression levels) with those of the plasmid vectors (no immunogenity or danger of virus recombination and relatively low costs).

Structure and production of MIDGE vectors—MIDGE minimalistic vectors contain only the expression cassette (promoter, coding sequence, and terminator/poly-A-site). They are smaller than plasmids by 50-80% and of linear covalently closed topology.

MIDGE vectors are made in a three step process, in which the expression cassette is cut out of a suitable plasmid. The resulting fragment of double stranded DNA is then covalently closed at both ends by loops of single stranded DNA oligonucleotides at both ends so that a continuous, covalently closed molecule results.

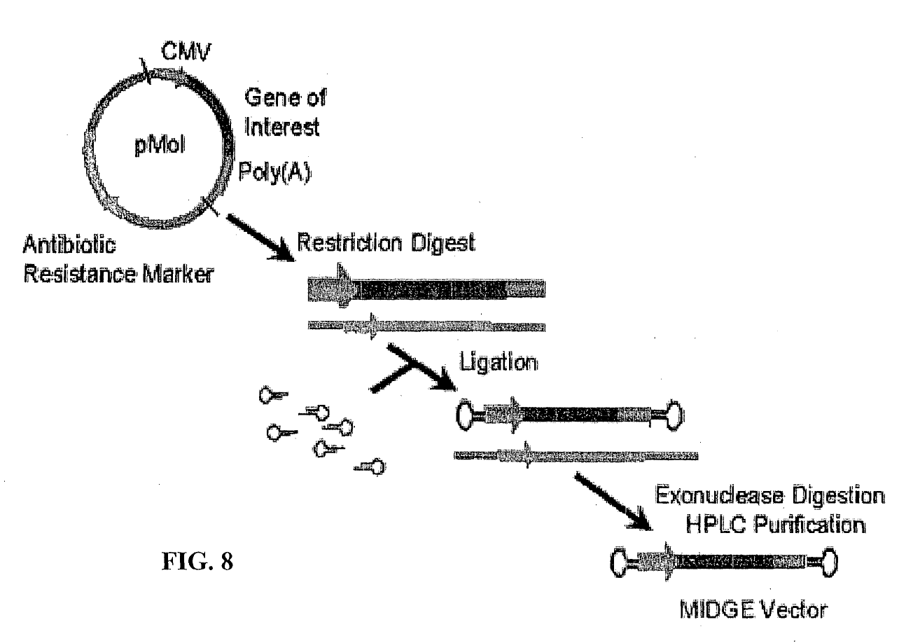

A graphical representation of MIDGE synthesis is shown in FIG. 8.

Advantages of the MIDGE-Technology:

MIDGE is safe—MIDGE vectors carry no sequence elements other than those needed for the medical objective of the gene transfer. Thus, there is no co-expression of antibiotic resistance genes from leaky bacterial promoters. Bacterial origins of replication and other sequence motives, which in bacteria tend to mediate integration or recombination, are completely absent in MIDGE. Therefore, integration into the genom of the host cell, of which the chances are very low already in plasmid DNA, is very unlikely to happen with MIDGE.

MIDGE is selective—The ease with which tissue specific ligands can be attached to the oligonucleotide loops makes the MIDGE-technology so versatile.

In effect it brings back some of the “viral” selectivity and gene transfer activity to the enhanced safety of the naked DNA approach.

Any vector will have to overcome two barriers before commencing gene expression: it has to get into the cell, and once within the cell, it has to reach the nucleus. A third, extracellular dimension of the problem is the delivery of the vector to the target cell population.

MIDGE allows for the attachment of tissue-specific ligands, e.g. peptides with known receptors on clinically relevant cell populations. The covalent attachement of a single decapeptide to the naked vector has led to surprisingly large effects in its immunogenecity, resulting in a profound difference in the induced immune response both in magnitude and quality. As a result, peptide-linked MIDGE vectors have been shown in a mouse model to be at least as effective as a DNA prime-vaccinia boost model, the best immunisation regime known so far.

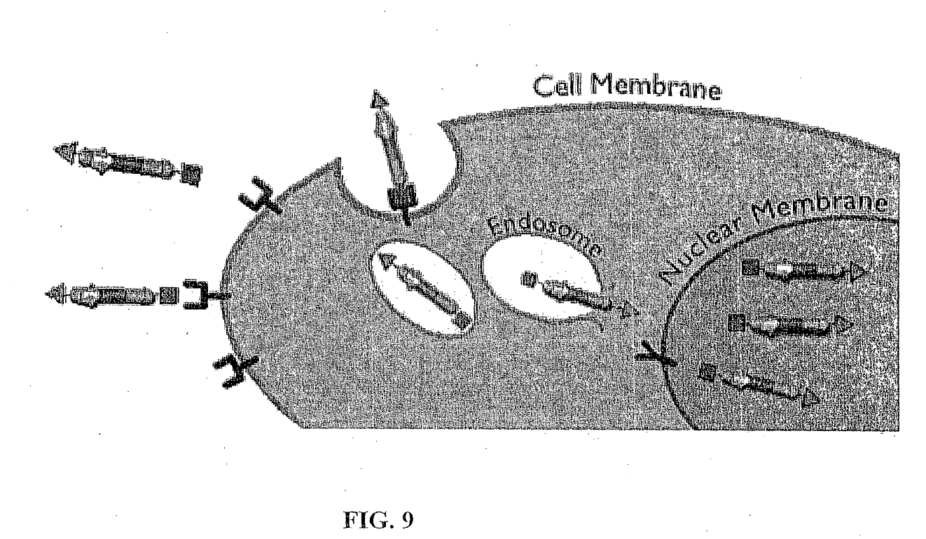

As shown in FIG. 9, MIDGE allows for the attachment of tissue-specific ligands for the active uptake of the entire vector molecule by receptor internalization into the cell cytoplasm. Nuclear import is mediated by nuclear localization sequences. A high level of gene expression is achieved.

MIDGE is non-immunogenic—In side-by-side comparison, MIDGE have shown several-fold increased expression of the encoded gene.

Summary of MIDGE vector advantages:

Every vector described above has its merit. Scientists at Mologen believe, that the MIDGE vectors features an ideal combination of the advantages of all other vectors:

MIDGE vectors are naked DNA, thus the dangers associated with viral vectors are of no concern.

At the same time, MIDGE vectors can be produce to target only specific cell types.

MIDGE vectors do not contain undesired sequences which could be hazardous to the patient.

No component of the MIDGE vector per se does elicit a significant immune response, thus avoiding undesired host reactions.

Immuno stimulatory sequence content (such as CpG) can be chosen according to the objective of the gene transfer.

MIDGE vectors are relatively simple and inexpensive to produce and are stable in handling and in the patient's body.

MIDGE vectors have been tested successfully in a number of clinical trials.

One feature of an embodiment of the invention resides broadly in a remedy for the reduction or suppression of the sensation of pain in higher animals, especially human beings, containing—with the exclusion of cells or cell lysates-expression constructs containing the POMC-sequence deleted of the coding regions for adrenocorticotropic hormone (ACTH) and beta-melanocyte stimulating hormone (β-MSH), which encode at least once for ββ-endorphin.

Another feature of an embodiment of the invention resides broadly in a remedy comprising single expression construct encodes for one, two or three β-Endorphin.

Yet another feature of an embodiment of the invention resides broadly in a remedy comprising an expression construct coding for corticotropin-releasing-factor (CRF).

Still another feature of an embodiment of the invention resides broadly in a remedy, wherein the βexpression construct is a plasmid or a linear, covalently closed expression construct.

A further feature of an embodiment of the invention resides broadly in a remedy that is applicable by injection.

Another feature of an embodiment of the invention resides broadly in a remedy where the DNA is complexed by polyethylenimine (PEI).

Yet another feature of an embodiment of the invention resides broadly in a remedy where the linear, covalently closed expression construct is modified with a peptide.

Still another feature of an embodiment of the invention resides broadly in a remedy where the linear, covalently closed expression construct is modified with a peptide comprising the nuclear localization sequence (NLS) of the large T-antigen of SV40.

Remedy according to claim 8, where the NLS peptide contains the amino acid sequence PKKKRKVEDPYC.

A further feature of an embodiment of the invention resides broadly in a remedy where the linear, covalently closed expression construct is conjugated to a cationic peptide of between 8 and 20 amino acids in length.

Another feature of an embodiment of the invention resides broadly in a vector for the production of an expression construct as a component of a remedy, containing the POMC-sequence deleted of the coding regions for adrenocorticotropic hormone (ACTH) and beta-melanocyte stimulating hormone (ββ-MSH), which encodes once for ββ-endorphin. ββ

Yet another feature of an embodiment of the invention resides broadly in a vector for the production of an expression construct as a component of a remedy, containing the POMC-sequence deleted of the coding regions for adrenocorticotropic hormone (ACTH) and beta-melanocyte stimulating hormone (β-MSH), which encodes at least twice for β-endorphin. βββ

Another feature of an embodiment of the invention resides broadly in a vector for the production of an expression construct as a component of a remedy, containing the POMC-sequence deleted of the coding regions for adrenocorticotropic hormone (ACTH) and beta-melanocyte stimulating hormone (β-MSH), which encodes three times for β-endorphin. βββ

Yet another feature of an embodiment of the invention resides broadly in a vector for the production of an expression construct as a component of a remedy, containing the desoxynucleic acid sequence of corticotropin releasing factor (CRF) (pMOK-CRF: Seq. ID 6). βββ

Still another feature of an embodiment of the invention resides broadly in a desoxyribonucleic acid sequence, containing one of the sequence tracts encoding β-endorphin from the pro-opiomelanocotrin gene (POMC), specifically the sequence shown in Seq. ID 1 (rPOMC 1×β-END).

A further feature of an embodiment of the invention resides broadly in a desoxyribonucleic acid sequence, containing two of the sequence tracts encoding (β-endorphin from the pro-opiomelanocotrin gene (POMC), specifically the sequence shown in Seq. ID 7 (rPOMC (β2×β-END).

Another feature of an embodiment of the invention resides broadly in a desoxyribonucleic acid sequence, containing three of the sequence tracts encoding β-endorphin from the pro-opiomelanocotrin gene (POMC), specifically the sequence shown in Seq. ID 2 (rPOMC 3×β-END).

All of the patents, patent applications, patent publications, or other publications, as well as the references and documents cited therein or in the bibliographies therewith, which were cited in the International Search Report dated Aug. 29, 2002, for International Application PCT/DE02/00583 and/or cited elsewhere, are hereby incorporated by reference as if set forth in their entirety herein as follows: Bauer et al., “Expression of Biologically Active Beta-Endorphin in K562 Cells,” Abstract SUN27, 31th Conference 2000 INRC, Jul. 16-18, 2000, XP002211427, Seattle, Wash., USA; WO 00 16800 A, Zoltan et al., Mar. 30, 2000; Tanelian et al., “Gene Therapy with Adenoviral β-Endorphin is Antinociceptive,” Anesthesiology, American Society of Anesthesiologists, Philadelphia, Pa.., USA, Bd. 85, Nr. 3A, September 1996 (09/1996)); Schafer et al., “Expression of corticotropin-releasing factor in inflamed tissue is required for intrinsic peripheral opioid analgesia,” Proceedings of the National Academy of Sciences of the United States of America, United States, Jun. 11, 1996, Bd. 93, Nr. 12, Jun. 11, 1996 (06/11/1996) Pages 6096-6100, XP002211428, ISSN: 0027-8424; Schafer et al., “Interleukin 1 beta and corticotropin-releasing factor inhibit pain by releasing opioids from immune cells in inflamed tissue,” Proceedings of the National Academy of Sciences of the United States of America, U.S., May 10, 1994, Bd. 91, Nr. 10, May 10, 1994 (05/10/1994), Pages 4219-4223, XP002211429, ISSN: 0027-8424; Database EMBL 'Online!, EMBL; Jan. 28, 1986 (1/28/1986), “EMBL:RNCRFR—Rat mRNA for corticotropin-releasing factor precursor (prepro-CRF)” retrieved from WWW.EBI.AC.UK, Database accession no. X03036, XP002211430; Database EMBL 'Online!, EMBL; Jul. 29, 1991 (7/29/1991), “EMBL:HSP0MC9—Human pro-opiomelanocortin (POMC) gene, exon 3.” retrieved from WWW.EBI.AC.UK, Database accession no. J00292, XP002211431; Chang et al., “Structural organization of human genomic DNA encoding the pro-opiomelanocortin peptide,” Proceedings of the National Academy of Sciences of the United States of America. United States Aug. 1980, Bd. 77, Nr. 8, August 1980 (08/1980), pages 4890-4894, XP001095590, ISSN: 0027-8424; Database EMBL 'Online!, EMBL; Jun. 13, 1985 (06/13/1985), “EMBL:RNPOMC3—Rat proopiomelanocortin (POMC) gene, exon 3.” retrieved from WWW.EBI.AC.UK, Database accession no. J00759, XP002211432; and EP 0 967 274 A (Mologen GMGH), Dec. 29, 1999 (12/29/1999).

The components disclosed in the various publications, disclosed or incorporated by reference herein, may be used in the embodiments of the present invention, as well as equivalents thereof.

Some examples of methods of and devices for performing reverse transcriptase which may possibly be utilized in at least one possible embodiment of the present invention may possibly be found in the following U.S. Pat. Nos. 6,608,188; 6,596,729; 6,593,337; 6,593,120; 6,589,768; 6,569,897; 6,555,686; 6,541,202; 6,514,979; 6,495,350; 6,492,515; 6,489,320; 6,489,098; 6,469,034; 6,465,171; 6,462,037; 6,451,532; 6,444,650; 6,440,735; and 6,423,718.

The appended drawings in their entirety, including all dimensions, proportions and/or shapes in at least one embodiment of the invention, are accurate and are hereby included by reference into this specification.

Some examples of universal primers which may possibly be utilized in at least one possible embodiment of the present invention may possibly be found in the following U.S. Pat. Nos. 6,207,372 and 5,882,856.

Some examples of methods of and devices for performing a polymerase chain reaction (PCR) which may possibly be utilized in at least one possible embodiment of the present invention may possibly be found in the following U.S. Pat. Nos. 6,596,492; 6,586,250; 6,586,233; 6,569,678; 6,569,627; 6,566,067; 6,566,052; 6,558,929; 6,558,909; 6,551,783; 6,544,782; 6,524,830; 6,518,020; 6,514,750; 6,514,706; 6,503,750; 6,493,640; 6,492,114; 6,485,907; and 6,485,903.

All, or substantially all, of the components and methods of the various embodiments may be used with at least one embodiment or all of the embodiments, if more than one embodiment is described herein.

Some examples of restriction enzymes which may possibly be utilized in at least one possible embodiment of the present invention may possibly be found in the following U.S. Pat. Nos. 6,495,325; 6,403,354; 6,258,539; 6,015,663; 5,955,369; 5,789,226; 5,470,732; 5,250,429; 5,179,016; 5,175,101; 5,165,933; 5,120,651; 4,960,707; 4,833,082; 4,808,531; 4,724,209; 4,668,631; and 4,542,099.

All of the patents, patent applications and publications recited herein, and in the Declaration attached hereto, are hereby incorporated by reference as if set forth in their entirety herein.

Some examples of methods of and devices for performing restriction digestion which may possibly be utilized in at least one possible embodiment of the present invention may possibly be found in the following U.S. Pat. Nos. 6,506,568; 6,046,039; 5,879,950; 5,595,870; and 3,953,609.

The corresponding foreign and international patent publication applications, namely, Federal Republic of Germany Patent Application No. 101 09 092.7, filed on Feb. 24, 2001, and DE-OS 101 09 092.7 and DE-PS 101 09 092.7, and International Application No. PCT/DE02/00583, filed on Feb. 19, 2002, having WIPO Publication No. WO02/067996, as well as their published equivalents, and other equivalents or corresponding applications, if any, in corresponding cases in Germany and elsewhere, and the references and documents cited in any of the documents cited herein, such as the patents, patent applications and publications, are hereby incorporated by reference as if set forth in their entirety herein.

Some examples of POMC which may possibly be utilized in at least one possible embodiment of the present invention may possibly be found in the following U.S. Pat. Nos. 6,603,058; 6,600,015; 6,589,958; 6,589,952; 6,586,456; 6,583,169; 6,583,143; 6,579,876; 6,579,852; 6,573,070; 6,566,332; 6,558,708; 6,551,795; 6,548,736; 6,541,478; 6,541,469; 6,541,244; 6,531,475; 6,525,056; and 6,521,636.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

Some examples of corticotropin which may possibly be utilized in at least one possible embodiment of the present invention may possibly be found in the following U.S. Pat. Nos. 6,589,947; 6,579,876; 6,548,509; 6,525,056; 6,521,636; 6,518,271; 6,515,005; 6,509,338; 6,469,041; 6,440,969; 6,403,599; 6,372,713; 6,365,589; 6,353,152; 6,353,103; 6,350,750; 6,348,571; 6,319,900; 6,300,360; and 6,271,380.

Some examples of CRF which may possibly be utilized in at least one possible embodiment of the present invention may possibly be found in the following U.S. Pat. Nos. 6,589,958; 6,586,456; 6,583,143; 6,541,469; 6,531,475; 6,518,271; 6,514,982; 6,500,839; 6,495,343; 6,482,836; 6,482,608; 6,448,265; 6,441,018; 6,440,960; 6,432,989; 6,399,315, 6,387,894; 6,384,039; 6,352,990; and 6,350,750.

The details in the patents, patent applications and publications may be considered to be incorporable, at applicant's option, into the claims during prosecution as further limitations in the claims to patentably distinguish any amended claims from any applied prior art.

Some examples of the restriction enzyme Eco31I which may possibly be utilized in at least one possible embodiment of the present invention may possibly be found in the following U.S. Pat. Nos. 6,599,703; 6,579,705; 6,451,563; 6,344,345; 6,303,308; 6,258,533; 6,190,889; 5,858,671; 5,658,736; 5,468,851; 5,436,150; 5,356,802; and 5,278,051.

Some examples of methods of and devices for performing gel electrophoresis which may possibly be utilized in at least one possible embodiment of the present invention may possibly be found in the following U.S. Pat. Nos. 6,582,574; 6,576,104; 6,569,306; 6,535,624; 6,406,602; 6,379,515; 6,301,377; 6,258,544; 6,197,173; 6,190,522; 6,127,134; 6,057,106; 6,043,025; 6,001,233; 5,989,400; 5,972,188; 5,938,909; 5,938,906; 5,916,427; and 5,904,826.

The abstract of the disclosure is submitted herewith as required by 37 C.F.R. §1.72(b). As stated in 37 C.F.R. §1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, the abstract is not intended to limit the claims in any manner and should not be interpreted as limiting the claims in any manner.

Some examples of methods of and devices for performing ethidium staining which may possibly be utilized in at least one possible embodiment of the present invention may possibly be found in the following U.S. Pat. Nos. 6,596,501; 6,593,465; 6,586,568; RE38,169; 6,573,300; 6,569,432; 6,558,898; 6,541,212; 6,514,697; 6,455,252; 6,451,578; 6,447,804; 6,399,307; 6,294,203; 6,270,962; 6,267,960; 6,262,252; 6,261,791; 6,261,789; and 6,258,939.

Some examples of methods of and devices for performing DNA injection which may possibly be utilized in at least one possible embodiment of the present invention may possibly be found in the following U.S. Pat. Nos. 6,319,224; 6,294,064; 6,214,804; 5,656,610; and 5,589,466.

Some examples of methods of and devices for performing gene or genetic injection which may possibly be utilized in at least one possible embodiment of the present invention may possibly be found in the following U.S. Pat. Nos. 6,525,030; 6,361,991; 6,090,790; 5,998,382; 5,697,901; 5,661,133; 5,273,525; 6,482,405; and 6,063,629.

The following U.S. Patents may possibly disclose structures or processes that may possibly be used in at least one possible embodiment of the present invention, and are hereby incorporated by reference as follows: U.S. Pat. Nos. 6,534,271, issued to Furste, et al. on Mar. 18, 2003; 6,451,593 issued to Wittig, et al. on Sep. 17, 2002; and 6,451,563 issued to Wittig, et al. on Sep. 17, 2002.

The following U.S. patents and foreign patent publications may possibly disclose structures or processes that may possibly be used in at least one possible embodiment of the present invention, as follows: U.S. Pat. No. 5,580,859; U.S. Pat. No. 5,584,807; U.S. Pat. No. 5,589,466; DE 198 54 946; DE 196 48 625; DE 198 26 758; EP 0686697; EP 0732395; WO 9626270; WO 9632473; WO 92/13963; WO 9313216; WO 94/12633; and WO 98/21322.

The following publications discuss genetic technology, such as gene therapy, DNA cloning, production, and manipulation thereof, and treatment and immunization of cells with DNA, and may possibly disclose structures or processes that may possibly be used in at least one possible embodiment of the present invention. These publications are incorporated by reference as follows: Eck, et al., 1996. Goodman & Gilman's The Pharmacological Basis of Therapeutics, Ninth Edition. McGraw-Hill, New York; Johnston, et al., 1993. Genetic Engineering, 15:225-236; “Immunization by Direct DNA Inoculation Induces Rejection of Tumor Cell Challenge” Wang et al., Human Gene Therapy 6:407-418 (Apr. 1995); “Identification of Wild-Type and Mutant p53 Peptides Binding to HLA-A2 Assessed by a Peptide Loading-Deficient Cell Line Assay and Novel Major Histocompatibilty Complex Class I-Peptide Binding Assay” Stuber et al., Eur. J. Immunol. 1994. 24:765-768; “Particle-Mediated Gene Transfer of Granulocyte-Macrophage Colony-Stimulating Factor cDNA to Tumor Cells: Implications for a Clinically Relevant Tumor Vaccine” Mahvi et al., Human Gene Therapy 7:1535-1543 (Aug. 20, 1996); “Ex Vivo Regulation of Specific Gene Expression by Nanomolar Concentration of Double-Stranded Dumbbell Oligonucleotides” Clusel et al., Nucleic Acids Research, 1993, vol. 21, No. 15, 3405-3411; “Dendritic Cells as Initiators of Tumor Immune Responses: A Possible Strategy for Tumor Immunotherapy?” Grabbe et al., Immunology Today, vol. 16, No. 3 1995, 117-121; “Sequence-Independent Inhibition of RNA Transcription by DNA Dumbbells and Other Decoys” Lim et al., Nucleic Acids Research, 1997, vol. 25, No. 3, 575-581; “A New Peptide Vector for Efficient Delivery of Oligonucleotides into Mammalian Cells” Morris et al., Nucleic Acids Research, 1997, vol. 25, No. 14, 2730-2736; “Improved Biological Activity of Antisense Oligonucleotides Conjugated to a Fusogenic Peptide” Bongartz et al., Nucleic Acids Research, 1994, vol. 22, No. 22, 4681-4688; “The Influence of Endosome-Disruptive Peptides on Gene Transfer Using Synthetic Virus-Like Gene Transfer Systems” Plank et al., The Journal of Biological Chemistry, vol. 269, No. 17, Apr. 29, pp. 12918-12924, 1994; “Linear Mitochondrial DNAs of Yeasts: Closed-Loop Structure of the Termini and Possible Linear-Circular Conversion. Mechanisms” Dinouel et al;., Molecular and Cellular Biology, Apr. 1993, pp. 2315-2323; “Heterologous Protection Against Influenza by Injection of DNA Encoding a Viral Protein” Ulmer et al., Science, vol. 259, Mar. 19, 1993, pp. 1745-1749; “Comparison of Organic Monolayers on Polycrystalline Gold Spontaneously Assembled from Solutions Containing Dialkyl Disulfides or Alkanethiols” Biebuyck et al., Langmuir 1994, 10, 1825-1831; “Regression of Established Murine Carcinoma Metastases Following Vaccination with Tumour-Associated Antigen Peptides” Mandelboim et al., Nature Medicine, vol. 1, No. 11, Nov. 1995, pp. 1179-1183; Kilisch et al. Covalently linked sequencing primer linkers (stinkers) for sequence analysis of restriction fragments. Gene vol. 44, pp. 263-270, Dec. 1986; Roberts, R J. Restricition and modification enzymes and their recognition sequences. vol. 13 Suppl. r165-r200, Dec. 1985; and Berger and Kimmel. Guide to molecular cloning techniques. Methods in Enzymology. vol. 52, Academic Press, Inc. New York. pp. 307-661, Dec. 1987.

The invention as described hereinabove in the context of the preferred embodiments is not to be taken as limited to all of the provided details thereof, since modifications and variations thereof may be made without departing from the spirit and scope of the invention.

What is claimed is:

1. Remedy for the reduction or suppression of the sensation of pain in higher animals, especially human beings, containing—with the exclusion of cells or cell lysates—expression constructs containing the POMC-sequence deleted of the coding regions for adrenocorticotropic hormone (ACTH) and beta-melanocyte stimulating hormone (β-MSH), which encode at least once for ββ-endorphin.

2. Remedy according to claim 1, wherein one single expression construct encodes for one, two or three β-Endorphin.

3. Remedy according to claim 1 or 2, which contains additionally an expression construct coding for corticotropin-releasing-factor (CRF).

4. Remedy according to claim 1, where the βexpression construct is a plasmid or a linear, covalently closed expression construct.

5. Remedy according to claim 4, which is applicable by injection.

6. Remedy according to claim 4, where the DNA is complexed by polyethylenimine (PEI).

7. Remedy according to claim 4, where the linear, covalently closed expression construct is modified with a peptide.

8. Remedy according to claim 7, where the linear, covalently closed expression construct is modified with a peptide comprising the nuclear localization sequence (NLS) of the large T-antigen of SV40.

9. Remedy according to claim 8, where the NLS peptide contains the amino acid sequence PKKKRKVEDPYC.

10. Remedy according to claim 4, where the linear, covalently closed expression construct is conjugated to a cationic peptide of between 8 and 20 amino acids in length.

11. Vector for the production of an expression construct as a component of a remedy, containing the POMC-sequence deleted of the coding regions for adrenocorticotropic hormone (ACTH) and beta-melanocyte stimulating hormone (ββ-MSH), which encodes once for ββ-endorphin. ββ

12. Vector for the production of an expression construct as a component of a remedy, containing the POMC-sequence deleted of the coding regions for adrenocorticotropic hormone (ACTH) and beta-melanocyte stimulating hormone (β-MSH), which encodes at least twice for β-endorphin. βββ

13. Vector for the production of an expression construct as a component of a remedy, containing the POMC-sequence deleted of the coding regions for adrenocorticotropic hormone (ACTH) and beta-melanocyte stimulating hormone (β-MSH), which encodes three times for β-endorphin. βββ

14. Vector for the production of an expression construct as a component of a remedy, containing the desoxynucleic acid sequence of corticotropin releasing factor (CRF) (pMOK-CRF: Seq. ID 6). βββ

15. Desoxyribonucleic acid sequence, containing one of the sequence tracts encoding β-endorphin from the pro-opiomelanocotrin gene (POMC), specifically the sequence shown in Seq. ID 1 (rPOMC 1×β-END).

16. Desoxyribonucleic acid sequence, containing two of the sequence tracts encoding β-endorphin from the pro-opiomelanocotrin gene (POMC), specifically the sequence shown in Seq. ID 7 (rPOMC β2×β-END).

17. Desoxyribonucleic acid sequence, containing three of the sequence tracts encoding β-endorphin from the pro-opiomelanocotrin gene (POMC), specifically the sequence shown in Seq. ID 2 (rPOMC 3×β-END).


SEQUENCE LISTING (10 sequences)

SEQ ID NO: 1 - DNA, 535 bp, Artificial
  rPOMC 1xbeta-END
atgaacttggagacaggcagccggggctcagagttcggcatgagcgcagtgagctgcggcaatgggaaacatgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctacggcggcttcatgacctccgagaagagccagacgcccctggtgacgctcttcaagaacgccatcatcaagaacgtgcacaagaagggccagtga

SEQ ID NO: 2 - DNA, 663 bp, Artificial
  rPOMC 3xbeta-END
atgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctacggcggcttcatgacctccgagaagagccagacgcccctggtgacgctcttcaagaacgccatcatcaagaacgtgcacaagaagggccagaagcgctacggcggcttcatgacctccgagaagagccagacgcccctggtgacgctcttcaagaacgccatcatcaagaacgtgcacaagaagggccagaagcgctacggcggcttcatgacctccgagaagagccagacgcccctggtgacgctcttcaagaacgccatcatcaagaacgtgcacaagaagggccagtga

SEQ ID NO: 3 - DNA, 708 bp, Rat
atgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctcctactccatggagcacttccgctggggcaagccggtgggcaagaagcggcgccctgtgaaggtgtaccccaatgtcgccgagaacgagtcggccgaggcctttcccctagagttcaagagggagctggaaggcgagcagcctgatggcttggagcacgtcctggagccggataccgagaaggccgacgggccctatcgggtggagcacttccgctggggcaacccgcccaaggacaagcgctacggcggcttcatgacctccgagaagagccagacgcccctggtgacgctcttcaagaacgccatcatcaagaacgtgcacaagaagggccagtga

SEQ ID NO: 4 - DNA, 615 bp, Artificial
  rPOMC - beta-END
atgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctcctactccatggagcacttccgctggggcaagccggtgggcaagaagcggcgccctgtgaaggtgtaccccaatgtcgccgagaacgagtcggccgaggcctttcccctagagttcaagagggagctggaaggcgagcagcctgatggcttggagcacgtcctggagccggataccgagaaggccgacgggccctatcgggtggagcacttccgctggggcaacccgcccaaggacaagcgctga

SEQ ID NO: 5 - protein, 12 aa, Simian virus 40
PKKKRKVEDPYC

SEQ ID NO: 6 - DNA, 1195 bp, Rattus norvegicus
  misc_feature: CRF cDNA sequence
aaactcagagcccaagtacgttgagaaactgaagagaaaggggaaaggcaaagaaaaggagaagagaaaggagaagaggaagaaaacctgcaggaggcatcctgagagaggtacctcgcagaacaacagtgcgggctcacctgccaagggaggagaagagagcgcccctaaacatgcggctgcggctgctggtgtccgcgggcatgctgctggtggctctgtcgccctgtctgccttgcagggccctgctgagcaggggatccgtctctggagcgccgcgggccccgcagccgttgaatttcttgcaaccggagcagccccagcaacctcagccgattctgatccgcatgggtgaagaatacttcctccgcctggggaacctcaacagaagtcccgctgctcggctgtcccccaactccacgcccctcaccgcgggtcgcggcagccgcccctcgcacgaccaggctgcggctaactttttccgcgtgttgctgcagcagctgcagatgcctcagcgcccgctcgacagcagcacggagctggcggaacgcggcgccgaggatgccctcggtggccaccagggggcgctggagagggagaggcggtccgaggagccgcccatctctctggatctcaccttccaccttctgagggaagtcttggaaatggccagggcagagcagttagctcagcaagctcacagcaacaggaaactgatggagattatcgggaaatgaaatgttgcgcttggccaaaacgattctgcatttagcacacaagtaaaaataaaaaatttaaaacacagtattctgtaccatactgcagctctgatatcatttgtttatttttatatagcttgaagcatagaagatgtacagggagagagcctatataccccttaattagcatgcacaaagtgtgtttctttgtagtaacaaaacagcgttatttgtattgttcacgcttagtttctatgtgcaaataagtgtctttatagcgatatcttaaagaaaatgtggatccaaggaggaaacctttaaaaaagcagatggaagtcacccagttgtttttatttggagacacagtgtaagagaattcattcttgaggggtggctaggacaaaatgtgtaagctctttgaatcaactttttcttgtaaatgtttcaataataaaacatctttctgatccttggtc

SEQ ID NO: 7 - DNA, 564 bp, Artificial
  rPOMC 2xbeta-END
atgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctacggcggcttcatgacctccgagaagagccagacgcccctggtgacgctcttcaagaacgccatcatcaagaacgtgcacaagaagggccagaagcgctacggcggcttcatgacctccgagaagagccagacgcccctggtgacgctcttcaagaacgccatcatcaagaacgtgcacaagaagggccagtga

SEQ ID NO: 8 - DNA, 936 bp, Homo sapiens
  Intron (847)..(933): beta-endorphin cDNA sequence
atgccgagatcgtgctgcagccgctcgggggccctgttgctggccttgctgcttcaggcctccatggaagtgcgtggctggtgcctggagagcagccagtgtcaggacctcaccacggaaagcaacctgctgaaggggatgggacaaaagaggcggtggcaagatcttagatgcccacgagtgccaagaaagcaggtgggcagacctgcctgtagggaggcctcgacgcttgacacgcccgacactgtgccctgtgtcctcggcgagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtga

SEQ ID NO: 9 - DNA, 87 bp, Rattus norvegicus
  misc_feature: beta-end sequence
ggcttcatgacctccgagaagagccagacgcccctggtgacgctcttcaagaacgccatcatcaagaacgtgcacaagaagggccag

SEQ ID NO: 10 - DNA, 87 bp, Homo sapiens
  misc_feature: beta-end sequence
ggtttcatgacctccgagaagagccagacgcccctggtgacgctgttcaaaaacgccatcatcaagaacgcctacaagaagggcgag